(19) United States
(12) Patent Application Publication
(10) Pub. No.: US 2007/0232695 A1
(43) Pub. Date: Oct. 4, 2007

(54) Gelled Periodontal Anesthetic Preparation

(75) Inventors: Mark Hirsh, Wellesley, MA (US); Jane C. Hirsh, Wellesley, MA (US); Mark W. Trumbore, Westford, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Collegium Pharmaceutical, Inc.

(21) Appl. No.: 11/534,552

(22) Filed: Sep. 22, 2006

Related U.S. Application Data
(63) Continuation-in-part of application No. 11/046,608, filed on Jan. 28, 2005.
(60) Provisional application No. 60/539,677, filed on Jan. 28, 2004.
(60) Provisional application No. 60/720,153, filed on Sep. 23, 2005.

(51) Int. Cl. A61K 31/325

(52) U.S. Cl. 514/537; 514/535

(57) ABSTRACT

A composition for anesthetizing oral or buccal tissues, especially periodontal pockets, is provided. The composition has a high concentration of topical anesthetic carried in a non-aqueous liquid vehicle containing a gelling agent. The anesthetics are optionally stabilized in the solution by ion-exchange complexation. The composition can anesthetize the gingivae for an extended period, such as 30 minutes or longer. Preferred anesthetics include tetracaine, benzocaine, butamben, and mixtures of these.

### RELATED APPLICATIONS

This application is a continuation-in-part of U.S. Ser. No. 11/046,608, filed Jan. 28, 2005 which claims the benefit of Ser. No. 60/539,677 filed on Jan. 28, 2004. This application also claims priority to U.S. Ser. No. 60/720,153, filed Sep. 23, 2005.

### FIELD OF THE INVENTION

The present invention is generally in the field of anesthetic formulations for periodontal applications.

### BACKGROUND OF THE INVENTION

Periodontitis is a disease caused by bacterial agents, frequently in combination with poor oral hygiene. Periodontal disease is caused mainly by the accumulation of bacteria (plaque). The destructive toxins and enzymes produced by these bacteria cause the ligaments holding the tooth in its socket to break down. As the ligaments degrade, the gingivae pull away from the tooth, resulting in a periodontal pocket between the tooth and gingivae. In these persistently infected pockets, bacteria lay down deposits and form biofilms, known as bacterial plaque. Plaque collects in these pockets, causing them to deepen. As plaque builds up and the pockets deepen, damage to the underlying supporting tissue continues. In advanced cases, periodontal scaling and root planning is used to interrupt the biofilms and allow normal tissue to regenerate. This procedure, which involves scraping the tooth and its roots below the gum line, is often painful, and local anesthesia is often necessary.

Local anesthetics are typically administered topically into or near the periodontal pocket. However, in some cases the anesthetic efficacy is low. In part, this may be because of the particular agents used. A popular currently used material is a gelled poloxamer solution containing an eutectic (oil form) of lidocaine and prilocaine, sold as “Oraqix®”, and described in U.S. Pat. No. 6,031,077 to Brodin et al., Brodin alleges that the composition is effective within 30 seconds, and lasts for about 20 minutes. It has been reported, however, that this composition becomes less effective the longer it is applied (Friskopp et al, J. Clin. Periodont. 28, 453-458 (2001)). This variable effectiveness may be due to the selection of the local anesthetics, as shown by Gurgius et al (Ann. Pharmacotherapy 36(4): 687-92, Apr. (2002)), and J Romsig et al. (Brit. J. Anaestheti. 83(4): 637-38 (1999)). In these studies, tetracaine exhibited a longer period of effectiveness than prilocaine/lidocaine. Moreover, the temperature sensitivity of the poloxamer requires the use of chilled poloxamer solutions in order to prevent gelation before insertion into the periodontal pocket. Finally, these formulations generally have a bitter taste.

Other delivery technologies have been used. U.S. Pat. No. 6,417,970 to Fanara et al., describes using phospholipids in organic solvent as carriers for antimicrobial drugs. U.S. Pat. No. 5,230,895 to Czarnecki et al describes forming a gel with monoglycerides. In each case, the organic phase disperses, causing the lipid to precipitate or gel on contact with periodontal fluid.

There exists a need for formulations which are well suited to deliver high concentrations of hydrophobic local anesthetics to the periodontal pockets and other oral or buccal tissues.

### SUMMARY OF THE INVENTION

Compositions containing one or more local anesthetics that are suitable for longer term anesthesia of oral tissues such as the periodontal pocket or buccal tissues have been developed. The compositions contain a substantially non-ionic and substantially non-aqueous (“NINA”) liquid vehicle and at least one local anesthetic dissolved or suspended in the vehicle. The anesthetic(s) can be absorbed on a high surface area material, such as an ion exchange resin or ion exchange polymer and dispersed in the liquid vehicle. The compositions may contain a polymeric viscosity modifying or gelling agent dissolved in the vehicle which will gel or thicken the solution on contact with a periodontal pocket or other intra-oral surface, or saliva, to form a anesthetic-delivering depot over a period of 20 minutes or more, preferably at least about 30 minutes, more preferably up to about 60 minutes. In one embodiment, the formulation contains a gelling agent, which is a mechanically-reversible thickening or gelling agent, which may be a shear-thinning liquid, sometimes called a “liquid gel”, so that the formulation can be applied via a syringe and gels in the periodontal pocket. Other formulations will contain a viscosity modifying agent, preferably a carbomer (polyacrylic acid USP; e.g., Carbopol™) polymer. The anesthetic solution in a nonionic solvent may coacervate or form microscopic gelled particles at the interface with aqueous liquid, such as saliva. In such cases, a secondary function of the gelling agent is to form a depot or precipitate in which the anesthetic is finely dispersed, and protected by the polymeric gelling agent from forming a simple second phase. This improves the ease of diffusion of the anesthetic from its phase to the gingivae. Other anesthetics can be dissolved in the vehicle, and absorbed directly by the tissue. In one embodiment, the compositions contain a combination of anesthetics with different absorption mechanisms.

### DETAILED DESCRIPTION OF THE INVENTION

Definitions

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “NINA” (an acronym for “non-ionic non-aqueous”) is used herein to designate a non-ionic, substantially non-aqueous, liquid, semi-solid or soft solid material used as a vehicle for delivery of active agent/carrier complexes or resinates (i.e., active agents bound or absorbed to a “carrier”), from any source, including animal, vegetable, mineral and synthetic. NINA vehicles are selected to be compatible with the skin for topical administration, and compatible with the gastrointestinal tract for oral administration.

As used herein, “carrier” refers to a particulate material which can complex one or more active agents. A preferred class of carrier is a “resin”, which includes polymeric materials used as carriers acting via ion exchange, absorption, etc. The term resin is sometimes used more broadly herein, unless otherwise distinguished, to include other particulate materials useable as carriers, including, but not limited to, charged inorganic materials.

As used herein, “complex” refers to covalent, ionic, hydrophobic and polar interactions. Examples of polar interactions include hydrogen bonding. Examples of hydrophobic interaction include Van der Walls forces, and pi stacking.

As used herein, a gel is either a dispersion of a colloidal solid (the dispersion phase) in a liquid, (the continuous phase) or a hydrogel wherein the continuous phase is an aqueous medium. The gel can be a solid or semi-solid material.

I. Formulations Containing Active Agent or Carrier-Bound Active Agent Compositions

A. Local Anesthetics

The local anesthetic may be a short acting local anesthetic, a long acting anesthetic, or a combination thereof. In one embodiment, the one or more anesthetics have a duration of action of at least 30 minutes, preferably at least 60 minutes. Extensive listings of local anesthetics, and discussion of their properties, can be found in standard reference books, for example “Martindale: The Complete Drug Reference”.

Suitable local anesthetics include, but are not limited to, aminoacylanilide compounds such as lidocaine, prilocaine, bupivacaine, levo-bupivacaine, ropivacaine, mepivacaine and related local anesthetic compounds having various substituents on the ring system or amine nitrogen; aminoalkyl benzoate compounds, such as procaine, chloroprocaine, propoxycaine, hexylcaine, tetracaine, cyclomethycaine, benoxinate, butacaine, proparacaine, butamben, isobuamben and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine; ketocaine, amethocaine, propanacaine, propipocaine, diamocaine cyclamate, etidocaine, levoxadrol HCl, octodrine, propoxycaine HCl, risocaine, rodocaine, salicyl alcohol, biphenamine HCl, ethyl chloride, oxethrazine, zolamine HCl, and combinations thereof. The anesthetic can be present as the free base or free acid form or as a pharmaceutically acceptable salt or labile ester. As is known, these forms will tend to interconvert in the presence of physiological fluids. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media such as ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The concentration of the anesthetics is variable. Combined anesthetic concentrations in the range (w/w) of about 12% to about 24% are preferred, as shown in the examples. Lower concentrations may be suitable if the anesthetic is especially potent, but are less preferred. Higher concentrations are possible if the entire system is storage stable, which can be determined experimentally.

In one embodiment, the formulation contains at least one water-soluble anesthetic and at least one lipid-soluble anesthetic. Because there is variability in the rate of delivery depending on the condition of the tissues, and on the rate of crevicular fluid introduction, having two or more different routes of absorption improves the likelihood of providing an effective amount of anesthetic.

For local anesthetics that are to be complexed to ion-exchange resins, the anesthetic is selected based on inclusion in the molecule of a group, such as an amino group, which will readily bind to a charged complexing agent such as an ion-exchange resin. Any active agent that bears an acidic or a basic functional group, for example, an amine, imine, imidazoyl, guanidine, piperdinyl, pyridinyl, quarternary ammonium, or other basic group, or a carboxylic, phosphoric, phenolic, sulfuric, sulfonic or other acidic group, can be bound to a resin of the opposite charge. Representative active agent agents are described in, for example, WO 98/18610 by Van Lengerich; U.S. Pat. No. 6,512,950 to Li et al. and U.S. Pat. No. 4,996,047 to Kelleher et al.

B. Non-Ionic, Non-Aqueous Vehicles

Formulations are prepared using a pharmaceutically acceptable non-ionic, non-aqueous vehicle composed of materials that are considered safe and effective and may be administered to an individual without causing undesirable biological side effects or unwanted interactions. The vehicle is a continuous phase in which the carrier is suspended, and in which excipients may be suspended or dissolved.

In principle, any liquid, semi-solid or soft solid (i.e., a material that could, for example, be swallowed, or used as a lotion base, or as an ointment base) can be used as a NINA vehicle if it is non-aqueous and does not contain an ion concentration sufficient to release the one or more active agents from the carrier. In one embodiment, the NINA is any material that is liquid at one or more of room temperature and body temperature and is sufficiently low in systemic toxicity and in local tissue damage.

Suitable NINA vehicles include, but are not limited to, plant oils such as sunflower oil, olive oil, peanut oil, corn oil, almond oil, cottonseed oil, sesame oil, soybean oil, canola, oil, castor oil, hydrogenated castor oil, and hydrogenated vegetable oil; animal oils such as fish liver oil and omega 3 lipids; organic solvents that are compatible with tissue, such as ethanol, propanol, t-butanol, glycerol, diethylene glycol, dipropylene glycol, liquid polyethylene glycols, polyethyleneglycol, and polypropylene glycol; lower molecular weight polyetherpolyols such as polyethylene glycols; mineral oils; silicon oil; semisolid materials, such as cholesterol, ergosterol, lanolin and lanolin alcohols, and petrolatum; lysolipids, phospholipds, crosprovidone; cyclomethinone; dibutyl phthalate; dibutyl sebacate; dimethicone; ethyl oleate; ethylene glycol palmitostearate; glycerin; glyceryl esters such as glycerol behenate, glyceryl monooleate, glyceryl monostearate, and glyceryl palmitostearate; isopropyl alcohol; isopropyl myristate; isopropyl palmitate; lecithin; magnesium stearate and zinc stearate; medium chain triglycerides, poloxomers and lower alkyl esters and ethers of these; polyethylene oxide; polyoxyethylene alkyl ethers; polyoxyethylene castor oil derivatives, polyoxyethylene sorbitan fatty acid esters; polyoxyethylene stearates; propylene carbonate; simethicone; sorbitan esters; higher molecular weight silicons, and other materials used as ointment bases; solid fatty materials, such as tallow and lard; waxes, such as paraffin, beeswax, carnuba wax, microcrystalline wax, and non-ionic and anionic emulsifying wax; hydrophobic resins and gums; fatty alcohols, such as stearoyl alcohol and stearyl alcohol; medium chain alkanes, such as octane, nonane, and decane; derivatives of alkanes, such as aldehydes, sulfonates, esters, ethers, ethoxylates; dioxane, dioxanone, ethyl acetate, ethyl lactate, methyl lactate, methyl ethyl ketone (MEK), dimethylformamide, dimethylsulfoxide, tetrahydrofuran, caprolactam, oleic acid, pyrrolidone, N-methyl pyrrolidone, and mixtures thereof.

A preferred class of liquid vehicles comprises alcohols, especially nontoxic polyols such as glycols (propylene glycol and higher) and glycerol. These include liquid polyethylene glycols and mixed polyalkylene glycols. Dipropylene glycol is a preferred polyol.

A non-aqueous phase has the advantage, for those anesthetics that will dissolve in it, of delivering them in molecular form to the surfaces of the tissue, where they can move directly into the hydrophobic cell membranes. Benzocaine and butamben are examples of anesthetics that will enter cells by this route. On the other hand, a lipid-insoluble anesthetic such as tetracaine HCl can be suspended in the non-aqueous fluid either as a fine powder, or preferably in an ion-exchange complex form, either complexed with a polymer having groups with the appropriate charge, or complexed with an ion exchange resin of the correct charge. As any of these forms encounter the tissues, the water-soluble molecules are displaced from the ion exchange groups by the evolved crevicular fluid. This will preferentially occur near the injured tissue surfaces.
i. Liquid Suspension

Complexed or non-complexed active agents can be dissolved or suspended in a NINA vehicle with the composition having (i) an absence of, or very low levels of, ionic ingredients, (ii) a low toxicity, and, optionally for oral administration, (iii) reasonable palatability. Liquid oral dosage forms include nonaqueous solutions, emulsions, suspensions, and solutions and/or suspensions reconstituted from non-effervescent granules, containing suitable solvents, emulsifying agents, suspending agents, diluents, sweeteners, coloring agents, and flavoring agents. Preservatives may or may not be added to the liquid oral dosage forms. Omission of preservatives is favored when possible due to possible allergic reactions to commonly used preservatives.

In preparing the liquid oral dosage forms, the active agent-carrier complexes are incorporated into an orally or topically acceptable NINA vehicle consistent with conventional pharmaceutical practices. The vehicle may include a suitable suspending agent. Known suspending agents include Avicel® RC-591 (a microcrystalline cellulose/sodium carboxymethyl cellulose mixture available from FMC), guar gum, alginate, carrageenan, pectin, xanthan, and the like. Such suspending agents are well known to those skilled in the art, and are suitable for use if they are compatible with a particular NINA vehicle. Suitability is readily tested by determining if the suspending agent prevents settling while not significantly affecting the controlled release properties of the coated active agent-loaded carriers.

A liquid suspension can be made by placing the active agent or the active agent-loaded particles into a liquid NINA vehicle. Surfactants may need to be added to allow dispersion of the particles in the oil.

For both oral and topical administration, the rate of release of the active agent can be controlled by controlling the water compatibility of the NINA vehicle. For example, a vehicle containing polyethylene glycol or propylene glycol will quickly being carrying water from the skin and the atmosphere to the active agent-loaded carriers, while a vehicle of isooctane will tend to prevent water access to the carriers until the vehicle has evaporated. A triglyceride vehicle such as olive oil or lard could have an even longer delaying effect, since water would penetrate slowly but the vehicle would not evaporate.
ii. Reconstitutable Dosage Units

Coated active agent-carrier complexes can be formulated into a granular material and packaged in a sachet, capsule or other suitable packaging in unit dose. Such granular material can be reconstituted at the time of use into a suitable NINA vehicle as described above. The granular material may contain excipients that facilitate the dispersion of the particles in the solvent or vehicle used. Formulations of this type have been disclosed in, for example, U.S. Pat. No. 6,077,532, and the manufacture of such unit doses and the use thereof are well known.

C. Carriers

The local anesthetic can be complexed to a carrier. Active agent/carrier complexes are generally prepared by complexing the active agent with a pharmaceutically acceptable carrier. The complex can be formed by reaction of a functional group on the active agent with a functional group on the carrier. Alternatively, the complex can be formed by the overall interaction of the active agent and the carrier, for example, via hydrophobic forces (Van Der Walls forces, pi stacking, etc.) or hydrogen bonding, or by entrapping the active agent within or on the carrier, for example following drying of an applied solution.

Suitable carriers include, but are not limited to, ion exchange resins; charged absorbents other than polymeric resins, including charged inorganic particulates such as silicates, aluminosilicates, and other inorganic particulates as well as particulate or crosslinked forms of natural polymers. Examples of derivatized natural polymer resins include but are not limited to, carboxymethyl cellulose, particulate forms of chitin, chitosan, and partially deacetylated chitin. Crosslinked forms of polymers such as glucomanans, galactomannans, galactoaminogylcans, glycosaminoglycans, hyaluronic acid, chondroitin sulfate, or polylysine can also be used as carrier.

In one embodiment, the binding resin is an ion exchange resin. For example, an active agent having a basic group such as an amino group can complex with an ion-exchange resin that bears an acidic group such as a sulfate or carboxylate group. Conversely, an active agent that has an acidic group can complex with an ion-exchange resin that bears a basic group. Active agents administered orally are released by exchanging with appropriately charged ions within the gastrointestinal tract. Active agents applied topically are released by fluids present on the skin, such as sweat, atmospheric moisture, or wound exudate, which either contain ions, or can liberate ions, when required, for release of the active agent from the carrier, from the skin or from separate ionic depots within the NINA vehicle.

Ion-exchange resins are water-insoluble materials, often cross-linked polymers, containing covalently bound salt forming groups in repeating positions on the polymer chain. The ion-exchange resins suitable for use in these preparations consist of a pharmacologically inert organic or inorganic matrix. The organic matrix may be synthetic (e.g., polymers or copolymers of acrylic acid, methacrylic acid, sulfonated styrene, sulfonated divinylbenzene), or partially synthetic (e.g., modified cellulose and dextrans). The ion exchange carrier can also be inorganic, e.g., silica gel, or aluminosilicates, natively charged or modified by the addition of ionic groups.

The covalently bound salt forming groups may be strongly acidic (e.g., phosphoric, sulfonic or sulfuric acid groups), weakly acidic (e.g., carboxylic acid), strongly basic (e.g., quaternary ammonium), weakly basic (e.g., primary amine), or a combination of these types of groups. Other types of charged groups can also be used, including any organic moiety that bears an acidic or a basic group, for example, an amine, imine, imidazoyl, guanidine, pyridinyl, quaternary ammonium, or other basic group, or a carboxylic phosphoric, phenolic, sulfuric, sulfonic, boric, boronic, or other acidic group.

In general, those types of ion-exchangers suitable for use in ion-exchange chromatography and for such applications as deionization of water are suitable for use in the controlled release compositions described herein. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975). The organic ion-exchange resins typically have exchange capacities below about 6 meq./g (i.e., 1 ionic group per 166 daltons of resin) and more commonly below about 5.5 meq./g.

Suitable ion-exchange resins include, but are not limited to, commercially available ion exchange resins such as Dowex® and other resins available from Rohm and Haas; Indion® resins available from Ion Exchange, Ltd. (India), Diaion® resins by Mitsubishi; BioRex® Type AG and other resins available from Dow Chemical; Amberlite® and Amberlyst® and other resins available from BioRad; Sephadex® and Sepharose® available from Amersham; resins by Lewatit, available from Fluka; Toyopearl® resins available from Toyo Soda; IONAC® and Whatman resins available from VWR; and Bakerbond® resins available from J T Baker.

Preferred ion exchange resins will be those supplied in grades known to be suitable for delivery of pharmaceuticals. Particular resins believed to be useful and approved include, without limitation, Amberlite® IRP-69 (Rohm and Haas), and INDION® 224, INDION® 244, and INDION® 254 (Ion Exchange (India) Ltd.). These resins are sulfonated polymers composed of polystyrene cross-linked with divinylbenzene.

The size of the ion-exchange particles is less than about 2 millimeters, preferably less than about 1000 microns, more preferably less than about 500 microns, more preferably less than about 150 micron (about 40 standard mesh). Commercially available ion-exchange resins (including Amberlite® IRP-69, INDION® 244 and INDION® 254 and numerous other products) are typically available in several particle size ranges, and many have an available particle size range less than 150 microns. The particle size is not usually a critical variable in terms of active agent release rate, but large particles can give a formulation a “gritty” feel, which is not desirable. When a formulation is a spray or an aerosol, the preferred particle size is less than about 100 microns, preferably less than about 50 microns, and more preferably less than about 20 microns. Particle size can be reduced before use, preferably before active agent loading, by milling, grinding and other known particle size-reduction techniques.

As used herein, the term “regularly shaped particles” refer to those particles which substantially conform to geometric shapes such as spherical, elliptical, and cylindrical. As used herein, the term “irregularly shaped particles” refers to particles excluded from the above definition, such as those particles with amorphous shapes with increased surface areas due to channels or distortions, or subsequent to grinding. For example, irregularly shaped ion-exchange resins of this type are exemplified by Amberlite® IRP-69 (supplied by Rohm and Haas), and to the active agent-resin complexes formed by binding active agents to these resins. Irregularly or regularly shaped particles may be used. The distinction between regularly shaped and irregularly shaped particles has been found by U.S. Pat. No. 4,996,047 to Kelleher et al to affect the degree of active agent loading required to prevent swelling and rupture of coating when loaded resins are placed in salt solutions, in the absence of fillers or impregnating agents, such as polyethylene glycol. They found that the critical value was at least 38% active agent (by weight in the active agent/resin complex) in irregular resins, and at least 30% by weight in regular resins.

Ion exchange resins have pores of various sizes, which expand the area available for active agent binding. The typical pore diameter is in the range of about 30 to 300 nanometers (nm), which is large enough for access by small-molecule active agents. For large active agents, such as proteins or nucleic acids, resins with larger pores, such as 500 to 2000 nm (0.5 to 2 micron), often called “macroreticular” or “macroporous”, are preferred.

Binding of active agent to a charged (ion-exchange) resin can be accomplished according to any of four general reactions. In the case of a basic active agent, these are: (a) resin (Na-form) plus active agent (salt form); (b) resin (Na-form) plus active agent (as free base); (c) resin (H-form) plus active agent (salt form); and (d) resin (H-form) plus active agent (as free base). Other pharmaceutically acceptable cations, especially K and Li, can be substituted for Na. All of these reactions except (d) have cationic by-products and these by-products, by competing with the cationic active agent for binding sites on the resin, reduce the amount of active agent bound at equilibrium. For basic active agents, stoichiometric binding of active agent to resin, i.e., binding an applied active agent molecule to essentially each binding site while having a very low level of active agent left in solution, is accomplished only through reaction (d).

Four analogous binding reactions can be carried out for binding an acidic active agent to an anionic exchange resin. These are: (a) resin (Cl-form) plus active agent (salt form); (b) resin (Cl-form) plus active agent (as free acid); (c) (as free base) plus active agent (salt form); and (d) resin (as free base) plus active agent resin (as free acid). Other pharmaceutically acceptable anions, especially Br, acetate, lactate and sulfate, can be substituted for Cl. All of these reactions except (d) have ionic by-products and the anions generated when the reactions occur compete with the anionic active agent for binding sites on the resin with the result that reduced levels of active agent are bound at equilibrium. For acidic active agents, stoichiometric binding of active agent to resin (as above) is accomplished only through reaction (d).

Active agent is bound to the resin by exposure of the resin to the active agent in solution via a batch process or a continuous process (such as in a chromatographic column). the active agent-resin complex thus formed is collected by filtration and washed with an appropriate solvent to insure removal of any unbound active agent or by-products. The complexes are usually air-dried in trays. Such processes are described in, for example, U.S. Pat. Nos. 4,221,778 to Raghunathan; 4,894,239 to Nonomura et al.; and 4,996,047 to Kelleher et al. Similar processes can also be used with ionic carriers other than ion exchange resins, such as silicates and other inorganic particles. However, these complexes may require collection by centrifugation or ultrafine filtration because of their small particle size.

The result of treating the ion exchange resin with a solution of active agent is an active agent-loaded particle with no coating. Such a particle can be used for active agent delivery with no additional treatment, especially in topical formulations. However, the loaded particles will typically be coated with one or more layers of materials to control the rate and location of release of active agent from the resin when the particles come in contact with a salt-containing aqueous solution, such as saliva, gastric juice or sweat.

D. Gelling and/or Viscosity Modifying Agents

The formulations may optionally contain a gelling agent and/or a viscosity modifying agent. It is believed that the relatively poor and transient anesthesia provided by current formulations is in part due to the rapid elution of the anesthetic by exchange with crevicular fluid. A gelled formulation may remain in place longer than an ungelled formulation, and even an increase in viscosity will retard elution. Since the anesthetic is continually leaving the periodontal pocket by diffusion into tissue, there is a limit to how long retention in the pocket is needed—a residence time in the range of about twenty minutes to an hour will typically be sufficient, although longer residence is preferred. A temporary gel or increase in viscosity is preferred, because anesthetic treatment is preferably completed while the patient is still in the periodontist's office. Hence there is no need for a gel to be long-lasting. Moreover, treatment may be repeated frequently, and there is typically insufficient time for the spontaneous “biodegradation” of precipitated polymers, for example, as described in U.S. Pat. No. 5,077,049 to Dunn, which are therefore not preferred. Hence, the “biodegradable” polymers as described in Dunn are excluded from the viscosity modifying agents of the present invention, unless used under conditions where they remain soluble or otherwise elutable from the sulcus within about a day or less.

It is difficult to place a dry powder in a periodontal pocket in a quantitative manner. It is also difficult to place a preformed article into the pocket, because each pocket differs in length, width and depth. Hence, administration as a liquid or as slurry is preferred. However, the volume of flow of gingival crevicular fluid in a periodontal pocket is difficult to measure but significant, and turnover of the sulcus (periodontal pocket) contents at least once a day in healthy tissue has been measured (J M Goodwin, Periodontology 2000 31, 43-54 (2003)). Significantly higher numbers are probably present in disease.

The objective is to retain the applied dose of anesthetic in the pocket in a simple, efficient manner. A first method is to implant a highly viscous solution. Viscosity can be provided by any convenient means; low fluid concentration in the implant, and presence of soluble or partially-soluble polymers in the implant, are preferred. A second method is to cause the implanted fluid to gel. One convenient method for inducing a gel state is to use a mixed solvent for a polymer, so that when the more fungible component evaporates or otherwise leaves the implant volume, or is replaced by water, the polymeric material gels. U.S. Pat. No. 4,745,160 to Churchill et al and U.S. Pat. No. 5,077,049 to Dunn et al describe such methods. In the context of the gingival pocket, the dilution of the applied material by secreted fluid can be a method for forming an implant.

E. Excipients

The formulation can contain one or more pharmaceutically acceptable excipients. The one or more excipients can be dissolved or dispersed in the non-ionic, non-aqueous vehicle. Suitable excipients include, but are not limited to, diluents, dispersing agents, solubilizing agents, surfactants, stabilizing agents, pH adjusting agents, flavoring agents, colorants, preservatives, and humectants. When the product forms a firm gel, pore-forming materials may give quicker access of the anesthetic to the tissue.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Nonaqueous Formulation Containing Benzocaine, Butamben, and Tetracaine

A formulation containing benzocaine, butamben, and tetracaine was dissolved and/or dispersed in dipropylene glycol. The elements of the formulation are shown in Table 1.

TABLE 1
Components of the Formulation

| Ingredient | Percent by weight |
|---|---|
| Dipropylene glycol | 80.6 |
| Benzocaine | 14.0 |
| Butamben | 2.0 |
| Tetracaine | 2.0 |
| Saccharin | 0.50 |
| Benzalkonium chloride | 0.555 |
| Cetydimethylammonium bromide | 0.005 |
| Flavor | 0.34 |
| TOTAL: | 100.0% |

The tetracaine preferably is present as a resinate (i.e., complexed onto an ion exchange resin). Sufficient resinate should be added to give an overall tetracaine concentration of about 2%. The exact amount of resinate required is determined for each batch of resinate, depending on the amount of tetracaine that is present per gram of resin.

The concentrate is mixed with a solution comprising xanthan dissolved in glycerol. The xanthan/glycerol solution is made by stirring while heating to 80 degrees C., and continued until a translucent uniform solution is obtained, after which the solution is allowed to cool. The concentration of the xanthan in glycerol is about 0.5% be weight. Between 1 and 5 parts by weight of the xanthan/glycerol solution are mixed with 10 parts of the above concentrate formulation. The viscosity of the mixture increases when mixed with a small amount, for example about 1 part by weight, of water.

The viscosity of the concentrate of Table 1 can be increased by gradually adding 1% by weight of carbomer (cross-linked polyacrylic acid) polymer, Carbopol™ type 934-P NF. The mixture is stirred at room temperature until the carbomer dissolves. The resulting viscous solution is stored at room temperature.

### Example 2

### Nonaqueous Benzocaine Formulation Suitable for Use as a Topical Anesthetic.

The concentrate composition is shown in Table 2.

TABLE 2

| Ingredient | Percent by weight |
|---|---|
| Dipropylene glycol | 78.74 |
| Benzocaine | 20.0 |
| Saccharin | 0.50 |
| Benzalkonium chloride | 0.555 |
| Cetydimethylammonium bromide | 0.005 |
| Flavor | 0.20 |
| TOTAL: | 100.0% |

The concentrate is mixed with 1% by weight of carbomer as above and stirred to obtain a uniform solution.

### Example 3

### Nonaqueous Tetracaine Formulation Suitable for Use as a Topical Anesthetic

Table 3 shows the composition of the concentrate.

TABLE 3

| Ingredient | Percent by weight |
|---|---|
| Dipropylene glycol | 82.6 |
| Tetracaine | 16.0 |
| Saccharin | 0.50 |
| Benzalkonium chloride | 0.555 |
| Cetydimethylammonium bromide | 0.005 |
| Flavor | 0.34 |

This viscosity of the concentrate can be increased by gradually adding 1% carbomer to a stirred solution and stirring until dissolution is complete.

We claim:

1. A composition for anesthetizing oral or buccal tissue, the composition comprising
a nonionic nonaqueous physiologically acceptable vehicle that is liquid at atmospheric pressure and 25° C. and a gel in the presence of an aqueous solution at 37° C., and
a viscosity modifying or gelling agent, and
an effective amount of one or more local anesthetic to provide anesthesia for at least 30 minutes at the tissue where it is applied, wherein the anesthetic is suspended or dissolved in the vehicle.

2. The composition of claim 1 wherein the thickening or gelling of the agent goes not require a change in temperature.

3. The composition of claim 1 wherein the local anesthetic comprises tetracaine or a pharmaceutically acceptable salt thereof in a concentration in the range of about 1% to about 20% by weight of the composition.

4. The composition of claim 1 wherein the anesthetic is a combination of anesthetics.

5. The composition of claim 4 wherein at least one of the combined anesthetics is selected from tetracaine, butamben and benzocaine.

6. The composition of claim 5 wherein the concentration of benzocaine is about 14%, the concentration of tetracaine is about 2%, and the concentration of butamben is about 2%, each percentage being by weight of the composition.

7. The composition of claim 1 where at least one anesthetic is present as a complex with an ion exchange material.

8. The composition of claim 7 where the complexed anesthetic is tetracaine hydrochloride.

9. The composition of claim 7 where the ion exchange material is selected from a polymer and a resin.

10. The composition of claim 1 characterized in that the anesthetic effect last for more than 30 minutes after an application time of about 1 minute of more.

11. The composition of claim 1 wherein the thickening or gelling agent comprises one or more materials selected from the group consisting of carbomer polyacrylic acid, xanthan, carboxymethylcellulose, polacrilin, polymethacrylate, crossarmelose, alkyl derivatives of cellulose, polyethylene oxides, poloxamers, povidone, polyvinyl alcohol, dextrin, dextran, and salts and labile esters thereof.

12. The composition of claim 12 wherein the thickening or gelling agent comprises carbomer.

13. The composition of claim 1 further comprising a taste-masking ingredient.

14. A method for anesthetizing a periodontal pocket during treatment thereof, the method comprising providing the composition of claim 1 into or adjacent to the tissue.
